(19) United States
(12) Patent Application Publication
(10) Pub. No.: US 2025/0391556 A1
(43) Pub. Date: Dec. 25, 2025

(54) MEDICAL DATA ACCESS METHOD AND MEDICAL DATA ACCESS SYSTEM

(71) Applicant: ASUSTeK COMPUTER INC., Taipei City (TW)

(72) Inventors: Ting-Hsuan Wang, Taipei City (TW); Min-Chang You, Taipei City (TW); Yu-Lun Chiang, Taipei City (TW); Shih-Wei Huang, Taipei City (TW); Hsiang-Hsuan Chen, Taipei City (TW); Fei-Wen Cheng, Taipei City (TW); Shih-Ni Chang, Taipei City (TW); Cathy Hui Tzu Chang, Taipei City (TW)

(73) Assignee: ASUSTeK COMPUTER INC., Taipei City (TW)

(21) Appl. No.: 19/198,144

(22) Filed: May 5, 2025

(30) Foreign Application Priority Data
Jun. 20, 2024 (TW) 113122916

(51) Int. Cl. G16H 40/63; G16H 10/60

(52) U.S. Cl. CPC G16H 40/63; G16H 10/60

(57) ABSTRACT

The present application relates to a medical data access system and a medical data access method thereof. The method includes the following steps. A user interface element of a front-end application is executed. The user interface element is linked to a data node of a front-end development library. Via the data node of the front-end development library, a medical data set of the data node is called from a back-end data system or a local cache device. According to the medical data set of the data node, target data is presented at the user interface element.

### CROSS-REFERENCE TO RELATED APPLICATION

This application claims the priority benefit of Taiwan application serial no. 113122916, filed on Jun. 20, 2024. The entirety of the above-mentioned patent application is hereby incorporated by reference herein and made a part of specification.

### BACKGROUND OF THE INVENTION

### Field of the Invention

The disclosure relates to a medical data access method and a medical data access system.

### Description of Related Art

In healthcare, efficient data management and integration are critical to improving patient care and operational efficiency. However, for developers, accessing and utilizing the data in a front-end application may be a complex and time-consuming task. Front-end developers face many challenges when using and integrating the large amounts of data generated by hospital systems (such as patient records, medical images, test results, etc.) during application. These challenges include the complexity of data structures and sources, the complexity of back-end API integration, and the fact that medical applications often require real-time data updates. Additionally, strict security requirements and the need to adhere to industry standards make front-end development environments more complex. Developers are responsible for ensuring data accuracy, security, and compliance. Front-end developers need to spend time and experience to overcome the above challenges, which results in poor front-end development efficiency and prevents front-end developers from focusing on designing the functions of front-end applications.

### SUMMARY OF THE INVENTION

The disclosure provides a medical data access method. The method includes the following steps. A user interface element of a front-end application is executed. The user interface element is linked to a data node of a front-end development library. Via the data node of the front-end development library, a medical data set of the data node is called from a back-end data system or a local cache device. According to the medical data set of the data node, target data is presented at the user interface element.

The disclosure provides a medical data access system including a storage device and a processor. The storage device records a plurality of commands. The processor is coupled to an input device and the storage device and configured to execute the commands to perform the following operations. A user interface element of a front-end application is executed. The user interface element is linked to a data node of a front-end development library. Via the data node of the front-end development library, a medical data set of the data node is called from a back-end data system or a local cache device. According to the medical data set of the data node, target data is presented at the user interface element.

Based on the above, in an embodiment of the invention, when the user interface element of the front-end application is executed, the corresponding medical data set may be called from the remote data system according to the data node linked to the user interface element. Then, according to the medical data set of the data node, the target data may be presented at the user interface element. Therefore, a front-end development library having a plurality of data nodes may serve as an intermediate layer between the front-end application and the back-end system to eliminate the complexity of calling the back-end API to allow front-end developers to focus on designing attractive and feature-rich applications.

In order to make the disclosure more comprehensible, embodiments are given below and described in detail with reference to the attached drawings.

### BRIEF DESCRIPTION OF THE DRAWINGS

FIG. 1 is a block diagram of a medical data access system shown according to an embodiment of the present application.

FIG. 2 is a flowchart of a medical data access method shown according to an embodiment of the present application.

FIG. 3 is a schematic diagram of a medical data access method shown according to an embodiment of the present application.

FIG. 4 is a schematic diagram of data classification shown according to an embodiment of the present application.

FIG. 5 is a flowchart of a medical data access method shown according to an embodiment of the present application.

### DESCRIPTION OF THE EMBODIMENTS

A portion of the embodiments of the invention is described in detail hereinafter with reference to figures. In the following, the same reference numerals in different figures should be considered to represent the same or similar elements. The embodiments are only a part of the invention, and do not disclose all possible implementation modes of the invention. Rather, the embodiments are merely examples of systems and methods within the scope of the invention.

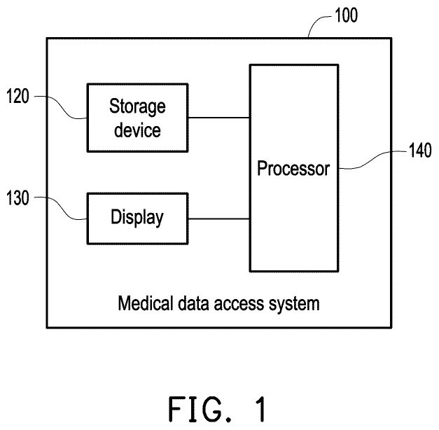

Please refer to FIG. 1. In the present embodiment, a medical data access system 100 may include a storage device 120, a display 130, and a processor 140. The medical data access system 100 may be, for example, a smart phone, a notebook computer, a tablet computer, a desktop computer, or various computer devices, etc. The present application is not limited thereto. In addition, in some embodiments, the medical data access system 100 may also be implemented by one or a plurality of electronic devices having computing capabilities.

The storage device 120 is used to store data and data such as a software module (such as an operating system, an application, a driver) for access by the processor 140 and may be, for example, any type of fixed or removable random-access memory (RAM), read-only memory (ROM), flash memory, hard disk, or a combination thereof. In some embodiments, the storage device 120 may include a local cache device.

The display 130 is, for example, a liquid-crystal display (LCD), a light-emitting diode (LED) display, an organic LED (OLED), or other types of displays, and the present application is not limited in this regard. In the present embodiment, the display 130 may display a user interface of a front-end application, such as a browser page and so on.

The processor 140 is coupled to the storage device 120 and the display 130. The processor 140 is, for example, a central processing unit (CPU), an application processor (AP), or other programmable general-purpose or special-purpose microprocessors, digital signal processors (DSPs), image signal processors (ISPs), graphics processing units (GPUs), or other similar devices, integrated circuits, and a combination thereof. In some embodiments, the processor 140 may access and execute the software module recorded in the storage device 120 to implement the medical data access method in an embodiment of the invention. The above software module may be broadly interpreted to mean command, command set, code, program code, program, application, software package, thread, procedure, function, etc., regardless of whether it is called software, firmware, middleware, microcode, hardware description language, or something else.

In an embodiment of the invention, a front-end development library as an intermediate layer between the back-end data system and the front-end application is provided. The front-end application may access the medical data set in the back-end data system via a plurality of data nodes in the front-end development library. The back-end data system includes a Hospital Information System (HIS). The plurality of data nodes in the front-end development library are responsible for managing and providing the data needed by the front-end application, and each data node may be regarded as a tool element in the front-end development library. In this case, front-end developers only need to interact with the data node to obtain or update data by triggering the data node. Therefore, front-end developers no longer need to deal with complex API requests and data management logic, and only needs to focus on the construction of interface and the optimization of user experience.

In an embodiment of the invention, the relevant data of the hospital information system (HIS) may be classified and constructed into a hierarchical data structure. Furthermore, the relevant data of the hospital information system (HIS) may be classified into a medical data set associated with each data node. From another perspective, the medical data sets corresponding to these data nodes may form a hierarchical data structure based on data classification.

In some embodiments, based on reference to international standard design terminology, such as the resource classification of Fast Healthcare Interoperability Resources (FHIR) or Epic's FHIR architecture design, etc., common HIS applications may be implemented via standardization and common semantics. In some embodiments, medical data classification may be implemented with reference to the data classification methods of health insurance systems of various countries. In this way, front-end developers may interact with these data nodes more intuitively and easily.

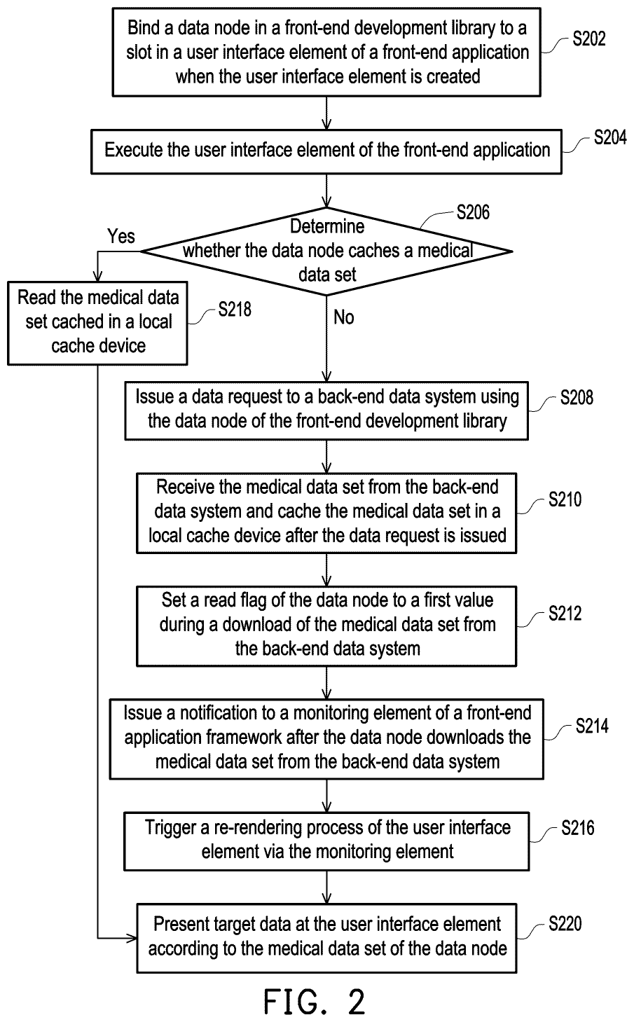

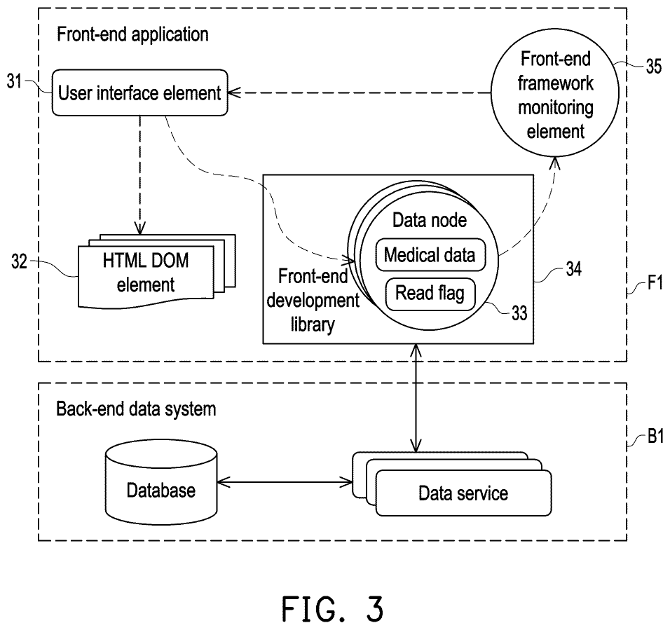

Please refer to FIG. 1 and FIG. 2 at the same time. The method of the present embodiment is applicable to the medical data access system 100. The detailed steps of the medical data access method of the present embodiment will be described below with each element of the medical data access system 100. In order to clearly describe possible implementation modes of the present application, the following description will be supplemented by FIG. 3. Please refer to FIG. 3 at the same time.

In step S202, when a user interface element 31 of a front-end application F1 is created, the processor 140 binds a data node 33 in a front-end development library 34 to a slot in the user interface element 31. Specifically, when creating the user interface element 31 using a front-end application framework (such as Vue3, React, or Angular), the processor 140 may control the front-end application framework to provide a program editing interface for binding the user interface element 31 to the data node 33. The processor 140 may bind the data node 33 to a slot in the user interface element 31 according to the input operation of front-end developers. In this way, by connecting the data node 33 and the user interface element 31, the medical data set corresponding to the data node 33 may be bound to the user interface element 31. The data node 33 encapsulates data acquisition, caching, and update logic.

In some embodiments, the first user interface element and the second user interface element may be linked to the same or different data nodes. When the first user interface element and the second user interface element are linked to the same data node, it means that the first user interface element and the second user interface element adopt the same medical data set.

In step S204, the processor 140 executes the user interface element 31 of the front-end application F1. The user interface element 31 is linked to the data node 33 of the front-end development library 34. Specifically, when the user interface element 31 of the front-end application F1 is executed, the processor 140 starts to render the user interface element 31 displayed on a web page, and generates a corresponding HTML Document Object Model (DOM) element 32. In other words, when the user interface element 31 is rendered, the corresponding HTML element is created and added to the DOM, thereby forming the web page content that the user sees in the browser.

In an embodiment of the invention, in response to rendering the user interface element 31, the processor 140 calls the medical data set of the data node 33 from a back-end data system B1 or a local cache device via the data node 33 of the front-end development library 34. That is, in response to rendering the user interface element 31, the processor 140 may call the data node 33 to which the user interface element 31 is connected. Then, the processor 140 determines whether the data node 33 contains a medical data set, that is, the processor 140 determines whether the medical data set corresponding to the data node 33 is cached in the local cache device.

In step S206, the processor 140 determines whether the data node 33 caches the medical data set. If the determination of step S206 is no, in step S208, the processor 140 issues a data request to the back-end data system B1 using the data node 33 of the front-end development library 34. That is, when the medical data set corresponding to the data node 33 is not yet downloaded, the data node 33 automatically issues a data request to the back-end data system B1.

In step S210, the processor 140 receives the medical data set from the back-end data system B1 and caches the medical data set in the local cache device after issuing the data request. It may be seen that the data node 33 may encapsulate the interaction logic with the back-end API and automatically process the data request and the response thereof. In this way, the processing of an API request by front-end developers may be simplified to reduce the complexity of interacting with the back-end.

In step S212, the processor 140 sets a read flag of the data node 33 to a first value during a download of the medical data set from the back-end data system B1. That is, during the period when the data node 33 receives and caches the medical data set, the processor 140 may set a read flag of the data node 33 to a first value. In response to the read flag being set to the first value, the processor 140 controls the user interface element 31 to display a “downloading” icon or text to let the user know whether the data is being downloaded. After the download of the medical data set of the data node 33 is completed, the processor 140 may set the read flag of the data node 33 to a second value.

In step S214, the processor 140 issues a notification to a monitoring element 35 of the front-end application framework after the data node 33 downloads the medical data set from the back-end data system B1. The monitoring element 35 is used to monitor data changes of the data node 33 and other data nodes. The monitoring element 35 is, for example, a Vue monitor. When the front-end development library 34 obtains or updates data from the back-end data system B1, the corresponding data node notifies the monitoring element 35.

In step S216, the processor 140 triggers a re-rendering process of the user interface element 31 via the monitoring element 35. That is, after the monitoring element 35 receives the notification issued by the data node 33, the monitoring element 35 triggers the re-rendering process of the user interface element 31 to ensure that the user interface displays the latest data and maintain the immediacy and accuracy of the front-end application. In the re-rendering process, the processor 140 may render the user interface element 31 according to the medical data set corresponding to the data node 33. In step S220, the processor 140 presents the target data at the user interface element 31 according to the medical data set of the data node 33.

Moreover, if the determination in step S206 is yes, it means that the medical data set corresponding to the data node 33 is cached in the local cache device. Therefore, in step S218, the processor 140 reads the medical data set cached in the local cache device. Next, in step S220, the processor 140 presents the target data at the user interface element 31 according to the medical data set of the data node 33.

In some embodiments, when the medical data set in the back-end data system B1 is updated, the processor 140 updates the medical data set of the data node 33 in the local cache device. The data node 33 may query the back-end data system B1 through polling whether there is data update. Alternatively, the back-end data system B1 may actively report the data update event to the data node 33. When the medical data set in the back-end data system B1 is updated, the data node 33 may issue a data request so that the back-end data system B1 provides a new medical data set to update the medical data set of the data node 33 in the local cache device.

In some embodiments, the processor 140 may execute another user interface element of a front-end application. The other user interface element is linked to another data node of the front-end development library, and the other data node is a child node of the data node. When the other data node is a child node of the data node and the data node caches the medical data set, the processor 140 may directly call the medical data set of the other data node from the local cache device. Specifically, based on a hierarchical data structure, the medical data set of the child node is a subset of the medical data set of the parent node. Therefore, if the data node as the parent node caches the corresponding medical data set, it means the medical data set of the child node thereof is also cached in the local cache device. Therefore, the processor 140 does not need to issue a data request to the back-end data system again for the medical data set of the child node.

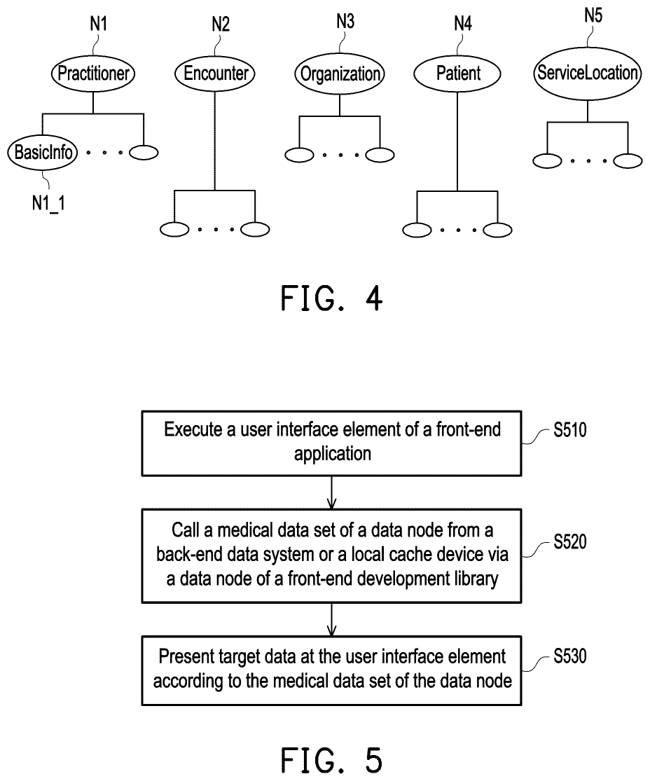

In some embodiments, the data node is one of a plurality of default data nodes in a hierarchical data structure. FIG. 4 is a schematic diagram of data classification shown according to an embodiment of the present application. In this example, the medical data related to the HIS system may be divided into five data nodes N1 to N5 at a first level. These five data nodes N1 to N5 are further subdivided into a plurality of data nodes at a second level. For example, the child node of the data node N1 includes a data node N1_1.

The data nodes N1 to N5 are data node “Practitioner”, data node “Encounter”, data node “Organization”, data node “Patient”, and data node “ServiceLocation” respectively. The medical data set corresponding to the data node “Practitioner” is the data related to people who are directly or indirectly involved in providing medical care or related services, such as the basic information and preferences of medical personnel, etc. The child node of the data node “Practitioner” includes the data node “BasicInfo”. The medical data set corresponding to the data node “BasicInfo” is the basic data of medical personnel.

The medical data set corresponding to the data node “Encounter” is data related to the medical environment, such as outpatient information, emergency information, home care information, etc. The medical data set corresponding to the data node “Organization” is data related to the hospital, such as hospital details and medical condition settings, etc. The medical data set corresponding to the data node “Patient” is data related to the patient, such as patient details and disease records, etc. The medical data set corresponding to the data node “ServiceLocation” is data related to the medical service location.

In this way, in the process of developing a front-end application, front-end developers may sequentially narrow the search scope of medical data sets based on the hierarchical data structure to quickly bind the user interface element to an appropriate data node. For example, when a physician identification code is to be presented via a user interface element, front-end developers may bind the user interface element to the data node N1_1, which is the data node “BasicInfo”. Front-end developers may bind this user interface element to the physician identification code in the medical data set of the data node N1_1 by creating the attribute path “XDS.Practitioner.BasicInfo.data.id”.

FIG. 5 is a flowchart of a medical data access method shown according to an embodiment of the present application. In step S510, a user interface element of a front-end application is executed. The user interface element is linked to a data node of a front-end development library. In step S520, via the data node of the front-end development library, a medical data set of the data node is called from a back-end data system or a local cache device. In step S520, according to the medical data set of the data node, target data is presented at the user interface element. However, each step in FIG. 5 is as described in detail above, and is not repeated herein. Moreover, the method of FIG. 5 may be used with the above exemplary embodiments, and may also be used alone, and the invention is not limited thereto.

Based on the above, in an embodiment of the invention, when the user interface element of the front-end application is executed, the corresponding medical data set may be called from the remote data system according to the data node linked to the user interface element. Then, according to the medical data set of the data node, the target data may be presented at the user interface element. Therefore, a front-end development library having a plurality of data nodes may serve as an intermediate layer between the front-end application and the back-end system to eliminate the complexity of calling the back-end API to allow front-end developers to focus on designing attractive and feature-rich applications.

In addition, the front-end development library of an embodiment of the invention may load the medical data set from the back-end data system according to actual needs, eliminating the need to preload huge and complete data sets. Data nodes may obtain data from the backend when needed and cache it locally to reduce repeated requests and improve application performance. In addition, it is ensured that front-end developers do not need to worry about the implementation details of the underlying API calls. This may effectively simplify the development process of front-end applications and make development more convenient. In addition, in an embodiment of the invention, the hierarchical data structure has high scalability, and front-end developers may seamlessly bind specific data to data nodes of the front-end development library. As a result, compatibility and easy integration of different data sources are ensured without compromising the core functionality of the front-end development library.

Although the disclosure has been described with reference to the above embodiments, it will be apparent to one of the ordinary skill in the art that modifications and variations to the described embodiments may be made without departing from the spirit and scope of the disclosure. Therefore, the scope of the disclosure shall be determined by the appended claims and its equivalent scope.

What is claimed is:

1. A medical data access method, comprising:
executing a user interface (UI) element of a front-end application, wherein the user interface element is linked to a data node of a front-end development library;
calling a medical data set of the data node from a back-end data system or a local cache device via the data node of the front-end development library; and
presenting target data at the user interface element according to the medical data set of the data node.

2. The medical data access method of claim 1, wherein the data node is one of a plurality of default data nodes of a hierarchical data structure.

3. The medical data access method of claim 1, wherein the step of calling the medical data set of the data node from the back-end data system or the local cache device via the data node of the front-end development library comprises:
determining whether the data node caches the medical data set;
issuing a data request to the back-end data system using the data node of the front-end development library in a case that the data node does not cache the medical data set; and
receiving the medical data set from the back-end data system and caching the medical data set in the local cache device after the data request is issued.

4. The medical data access method of claim 3, wherein the step of calling the medical data set of the data node from the back-end data system or the local cache device via the data node of the front-end development library further comprises:
reading the medical data set cached in the local cache device in a case that the data node caches the medical data set.

5. The medical data access method of claim 1, further comprising:
updating the medical data set of the data node in the local cache device when the medical data set in the back-end data system is updated.

6. The medical data access method of claim 1, further comprising:
issuing a notification to a monitoring element of a front-end application framework after the data node downloads the medical data set from the back-end data system; and
triggering a re-rendering process of the user interface element via the monitoring element.

7. The medical data access method of claim 6, further comprising:
setting a read flag of the data node to a first value during a download of the medical data set from the back-end data system.

8. The medical data access method of claim 1, further comprising:
binding the data node in the front-end development library to a slot in the user interface element when the user interface element of the front-end application is created.

9. The medical data access method of claim 1, wherein the back-end data system comprises a hospital information system (HIS).

10. The medical data access method of claim 1, further comprising:
executing another user interface element of the front-end application, wherein the other user interface element is linked to another data node of the front-end development library, and the other data node is a child node of the data node; and
calling a medical data set of the other data node directly from the local cache device when the other data node is the child node of the data node and the data node caches the medical data set.

11. A medical data access system, comprising:
a storage device recording a plurality of commands; and
a processor connected to the storage device and configured to execute the commands to:
execute a user interface (UI) element of a front-end application, wherein the user interface element is linked to a data node of a front-end development library;
call a medical data set of the data node from a back-end data system or a local cache device via the data node of the front-end development library; and
present target data at the user interface element according to the medical data set of the data node.
